(19) United States
(12) Patent Application Publication
(10) Pub. No.: US 2021/0220107 A1
(43) Pub. Date: Jul. 22, 2021

(54) PATTERNS FOR ENERGY DISTRIBUTION, METHODS OF MANUFACTURE THEREOF AND ARTICLES COMPRISING THE SAME

(71) Applicants: SHARKLET TECHNOLOGIES, INC., Aurora, CO (US); UNIVERSITY OF FLORIDA RESEARCH FOUNDATION, INC., GAINESVILLE, FL (US)

(72) Inventors: Anthony B. Brennan, Gainesville, FL (US); Ryan Eugene Stoneberg, Aurora, CO (US); Kevin Lee Ballard, Aurora, CO (US)

(21) Appl. No.: 17/273,846

(22) PCT Filed: Sep. 4, 2019
(86) PCT No.: PCT/US2019/049416

§ 371 Date: Mar. 5, 2021

Related U.S. Application Data
(60) Provisional application No. 62/728,396, filed on Sep. 7, 2018.

(51) Int. Cl. A61F 2/00

(52) U.S. Cl. CPC A61F 2/0077; A61F 2002/0081

(57) ABSTRACT

Disclosed herein are patterns and pattern distributions that reduce gloss and glare in surfaces that are textured. Patterns of different sizes and orientations are disclosed herein.

### BACKGROUND

This disclosure relates to patterns for energy distribution, methods of manufacture thereof and articles comprising the same.

Texturing of surfaces has been developed for controlling bioadhesion, for flow control of fluids in contact with the textured surface, and for a variety of other reasons. FIG. 1 depicts a surface texture 100 that can be used for controlling bioadhesion as well as for flow control. The texture comprises a plurality of features 111 that are arranged to have edges 130 that parallel to each other in at least one direction. As can be seen in the FIG. 1, the features are arranged in patterns (encompassed by the dotted lines) 102 that are repeated across the textured surface.

The arrangement of the plurality of features with edges that are parallel to each other in repeating patterns promotes a large amount of constructive interference of any light that is incident on the surface. This constructive interference produces a tremendous amount of gloss and glare that may be distracting to the viewer.

FIGS. 2 and 3 depict another textured surface 100 that contains repeating patterns where some of the patterns are oriented at a different angles when compared with some of the other patterns. In the FIG. 2, the patterns in the 4 quadrants (1, 2, 3 and 4 respectively) are oriented different directions with respect to each other. The axis AA′ indicates the axis of orientation of the pattern in a first quadrant, while the pattern BB′ indicates the orientation of the pattern in a neighboring quadrant. From the FIG. 2, it may be seen that the axis AA′ oriented orthogonally to the axis BB′. The patterns in quadrants 1 and 3 are therefore oriented at right angles with respect to the patterns in the quadrants 2 and 4. This orientation of the patterns is used to control fluid flow on a surface in a particular direction because the length of the tortuous path that a fluid has to flow in order to get across the pattern is increased tremendously. By orienting the patterns in mutually perpendicular directions, the fluid flow in one direction is obstructed by the patterns in a neighboring quadrant thus minimizing fluid flow across the pattern.

In the FIG. 2, however, it may still be seen that a plurality of patterns are oriented in a particular direction. Thus when light is incident upon the surface, constructive interference between the features that are arranged in one quadrant produces gloss and glare that are displeasing to the viewer and uncomfortable to the eyes of the viewer.

FIG. 3 also shows a textured surface 100 that comprises a plurality of patterns that are oriented with respect to one another. In the FIG. 3, there are three different orientations of the features in the patterns, P, M and N respectively. This orientation can be advantageously used to control and direct flow of fluid by varying the pattern orientation. However, even in this embodiment, there is a long range order amongst the features of the patterns that results in undesirable glare and gloss. The long range order in the FIG. 3 may be seen along the axis XX′, YY′ and AA′. The long range order in each of these directions results in constructive interference (in each of these directions) that produces glare and gloss that is displeasing to the viewer.

It is therefore desirable to produce textured surfaces where the bioadhesive properties can be retained, while at the same time minimizing gloss and glare.

### SUMMARY

Disclosed herein is an article comprising a pattern comprising a first plurality of spaced features; the spaced features arranged in a plurality of groupings; the groupings of features comprising repeat units; the spaced features within a grouping being spaced apart at an average distance of about 1 nanometer to about 500 micrometers; each feature having a surface that is substantially parallel to a surface on a neighboring feature; each feature being separated from its neighboring feature; and wherein the groupings of features being arranged with respect to one another so as to define a tortuous pathway; and further wherein

a) the longitudinal axis of neighboring features are not parallel to one another and are inclined at varying angles of 5 to 85 degrees with respect to one another;

b) the patterns are grouped together in groups of 4 to 20 with each neighboring group having a different axis of orientation;

c) opposing surfaces of the article each have the texture, where the texture on one surface is oriented differently from the texture on the opposing surface;

d) the features of the texture have roughened surfaces; and/or

e) the features contain fillers that can absorb visible light.

Disclosed herein too is a method comprising disposing on a surface a pattern comprising a first plurality of spaced features; the spaced features arranged in a plurality of groupings; the groupings of features comprising repeat units; the spaced features within a grouping being spaced apart at an average distance of about 1 nanometer to about 500 micrometers; each feature having a surface that is substantially parallel to a surface on a neighboring feature; each feature being separated from its neighboring feature; and wherein the groupings of features being arranged with respect to one another so as to define a tortuous pathway; and further wherein

a) the longitudinal axis of neighboring features are not parallel to one another and are inclined at varying angles of 5 to 85 degrees with respect to one another;

b) the patterns are grouped together in groups of 4 to 20 with each neighboring group having a different axis of orientation;

c) opposing surfaces of the article each have the texture, where the texture on one surface is oriented differently from the texture on the opposing surface;

d) the features of the texture have roughened surfaces; and/or

e) the features contain fillers that can absorb visible light.

### BRIEF DESCRIPTION OF THE FIGURES

FIG. 1 depicts patterns on a textured surface in which the features are arranged in a repeating fashion across a surface;

FIG. 2 depicts another textured surface that contains repeating patterns where some of the patterns are oriented at a different angles when compared with some of the other patterns;

FIG. 3 depicts another textured surface that contains repeating patterns where some of the patterns are oriented at a different angles when compared with some of the other patterns;

FIG. 4A depicts one arrangement of features on a surface that can be used to control bioadhesion;

FIG. 4B depicts another arrangement of features on a surface that can be used to control bioadhesion;

FIG. 4C depicts another arrangement of features on a surface that can be used to control bioadhesion;

FIG. 4D depicts another arrangement of features on a surface that can be used to control bioadhesion;

FIG. 5 depicts the basic repeat unit that forms the texture shown in the FIG. 4A;

FIG. 6 depicts that the axis AA′ of one element of the pattern and the axis AB′ of the neighboring element are inclined at an angle θ;

FIG. 7 depicts a texture obtained by reproducing the repeat pattern of the FIG. 6 in a series of rows;

FIG. 8 depicts patterns where elements labelled 1 and 2 in pattern A are separated by a different angle that the elements labelled 1 and 2 in neighboring pattern B;

FIG. 9A shows the texture on the first surface of a film;

FIG. 9B shows the texture on a second surface of the film of the FIG. 9A, where the second surface is opposed to the first surface;

FIG. 9C depicts a pattern on a first side of a film where the axis of the pattern represented by line XX′ is vertical;

FIG. 9D depicts a pattern on a second side of the film of the FIG. 9C where the axis of the pattern represented by the line XX′ is horizontal. The second side of the film is opposed to the first side;

FIG. 9E depicts two overlapping patterns on opposing surfaces of a film having their axis inclined at 90 degrees;

FIG. 10 depicts another embodiment where neighboring groups of patterns (Grain 1 and Grain 2) are rotated with respect to each other;

FIG. 11 depicts an arrangement of groups of patterns to control glare. This arrangement leaves a variety of open spaces between the grains that need to be filled in;

FIG. 12 shows one manner of filling such open spaces with partial patterns;

FIG. 13 depicts another texture where the spaces between Grains 1 and 6 and between Grains 1 and 2 are filled with partial patterns and triangles;

FIG. 14A depicts another texture where patterns having different sizes may be used; FIG. 14B is just an enlarged version of a portion of FIG. 14A; and

FIG. 15 depicts how varying feature density may be used to reduce gloss.

### DETAILED DESCRIPTION

Disclosed herein is an article comprising a surface that has a texture with reduced gloss and glare as a result of which it is easier on the eyes of the viewer. The texture is designed to reduce constructive interference while at the same time providing a user with the ability to control bioadhesion, flow control, and the like. The texture comprises patterns that comprise a plurality of features that include one or more of the following a) the longitudinal axis of neighboring features are not parallel to one another and are inclined at varying angles of 5 to 85 degrees with respect to one another; b) the patterns are grouped together in groups of 4 to 20 with each neighboring group having a different axis of orientation; c) opposing surfaces of the article each have the texture, where the texture on one surface is oriented differently from the texture on the opposing surface; d) the features of the texture have roughened surfaces; e) the features contain fillers that can absorb visible light. Groups of patterns are also referred to herein as grains; or a combination thereof.

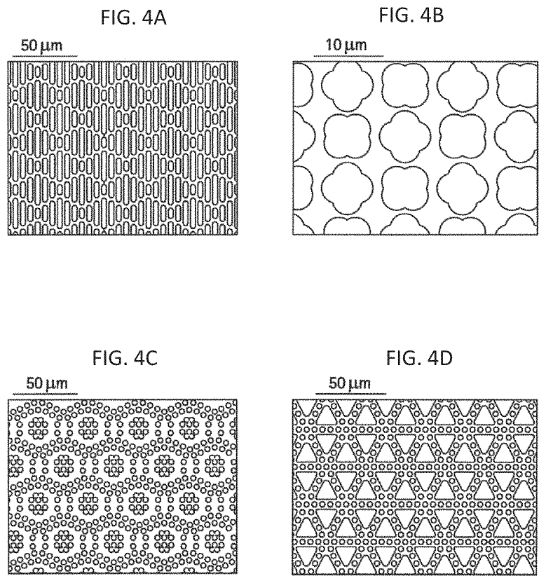

The basic surface texture that is used control bioadhesion is shown in the FIGS. 4A-4D. The surface texture comprises a set of features that are repeated across the surface. The texture can comprise a plurality of spaced features, where the features are arranged in a plurality of groupings (also referred to herein as a “pattern”); the groupings of features being arranged with respect to one another so as to define a tortuous pathway when viewed in a first direction. When viewed in a second direction, the groupings of features are arranged to define a linear pathway.

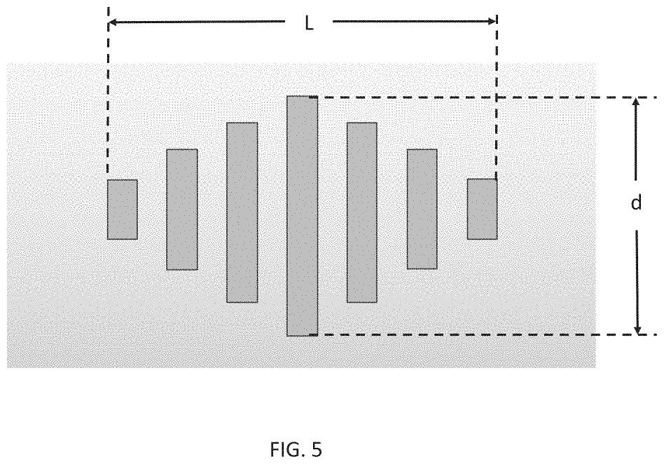

FIG. 5 depicts the basic repeat unit that forms the texture shown in the FIG. 4A. The basic repeat unit comprises a plurality of elongated spaced features that are parallel to each other, but that when aligned as seen in the FIG. 4A, define a sinusoidal pathway when viewed in a first direction. The pathway when viewed in the first direction may also be represented by a spline function. In one embodiment, when viewed in a second direction, the pathway between the features may be non-linear and non-sinusoidal. In other words, the pathway can be non-linear and aperiodic. In another embodiment, the pathway between the features may be linear but of a varying thickness. The plurality of spaced features may be projected outwards from a surface or projected into the surface. In one embodiment, the plurality of spaced features may have the same chemical composition as the surface. In another embodiment, the plurality of spaced features may have a different chemical composition from the surface. In other words, the features may be bonded to the surface to adjust the surface energy. In another embodiment, the features and the surface may be monolithic (i.e., they form one undivided article).

In an embodiment, the surface texture comprises a plurality of identical patterns; each pattern being defined by a plurality of spaced apart features attached to or projected into the first surface where at least one spaced apart feature having a dimension of about 1 nanometer to about 1 millimeter, preferably 5 nanometers to 500 micrometers, and more preferably 100 nanometers to 50 micrometers. In an embodiment, the plurality of spaced features has a similar chemical composition to the surface. In another embodiment, the plurality of spaced features has a different chemical composition from the composition of the surface. The plurality of spaced features is applied to the surface in the form of a coating. The patterns on the article have an engineered roughness index (ERI) of about 2 to about 30, preferably 5 to 25.

The plurality of features each have at least one neighboring feature having a substantially different size or geometry, wherein each pattern has at least one feature which is identical to a feature of a neighboring pattern and shares that feature with the neighboring pattern. The average spacing between adjacent spaced apart features is about 1 nanometer to about 1 millimeter in at least a portion of the first surface and/or the second surface (which is opposed to the first surface and in contact with it). The plurality of spaced apart features are represented by a periodic function since the features in the patterns are equidistant from each other. The equidistant spacing results in constructive interference when the surface is irradiated with visible light. This also results in the production of gloss, which can hurt the eye of the viewer.

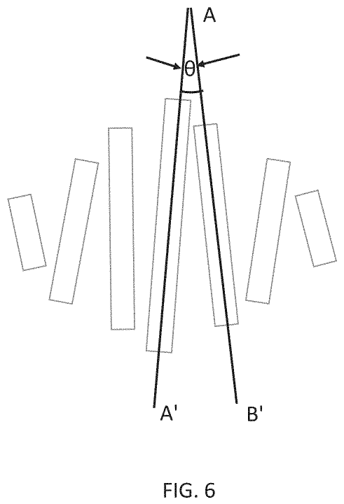

In order to reduce the gloss, the individual features of the basic unit may be inclined at angles to one another, such that these features are no longer parallel to each other. FIG. 6 depicts one such structure. The individual features are rectangular in geometry (when viewed from the top), but an axis of each feature is inclined at an angle of 5 to 85 degrees with an axis of a neighboring feature. In the FIG. 6, it may be seen that the axis AA′ of one element of the pattern and the axis AB′ of the neighboring element are inclined at an angle θ that can vary from 3 to 88 degrees, preferably 4 to 25 degrees, and more preferably 5 to 20 degrees. It is desirable for the features to avoid contact with one another. The angle of inclination depends upon the distance between the individual features as well as the length of the individual features. The angle of inclination may be increased as the distance between the individual features is increased. The angle of inclination may also be increased as the aspect ratio of the individual features is decreased.

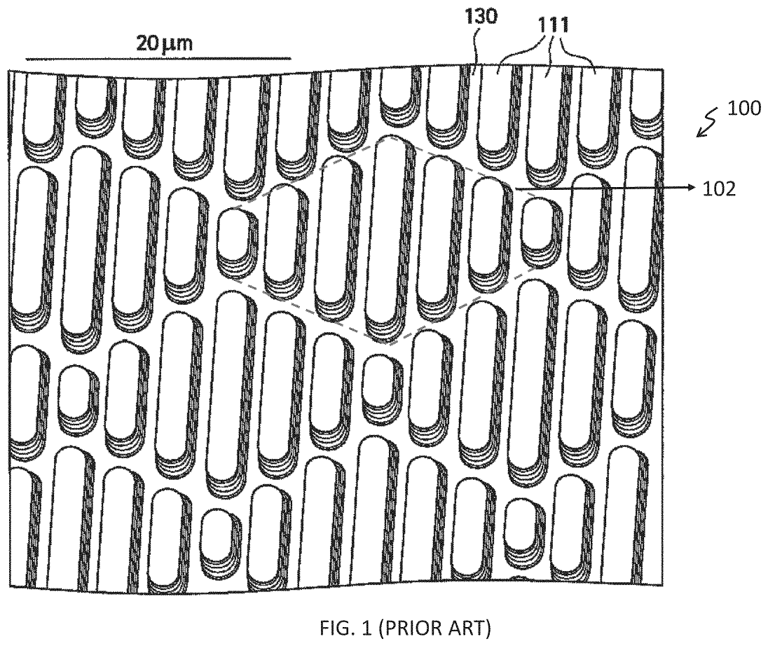

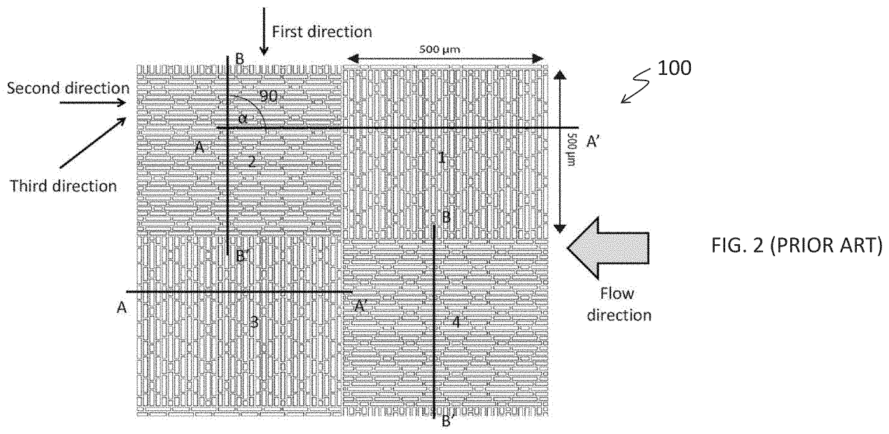

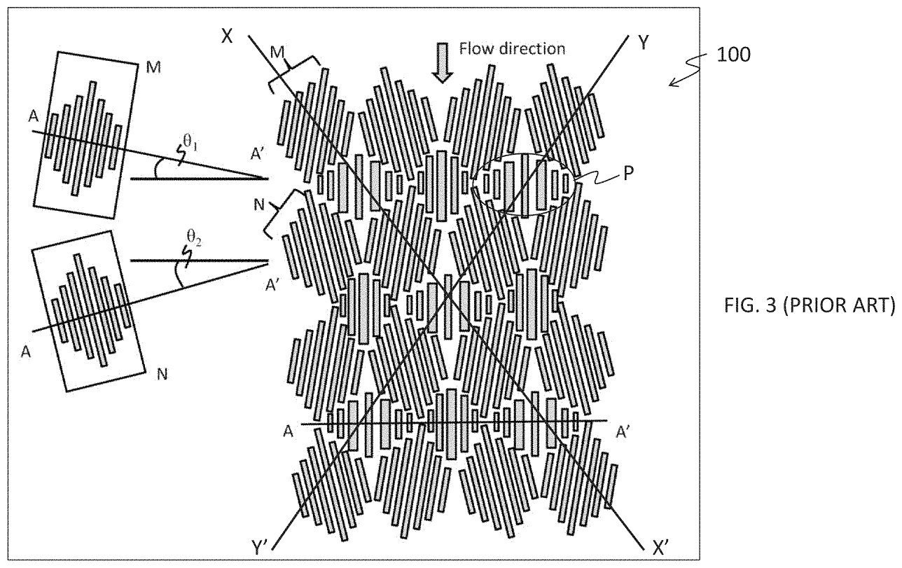

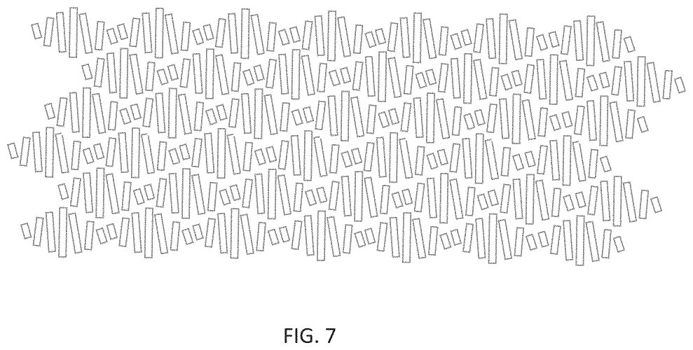

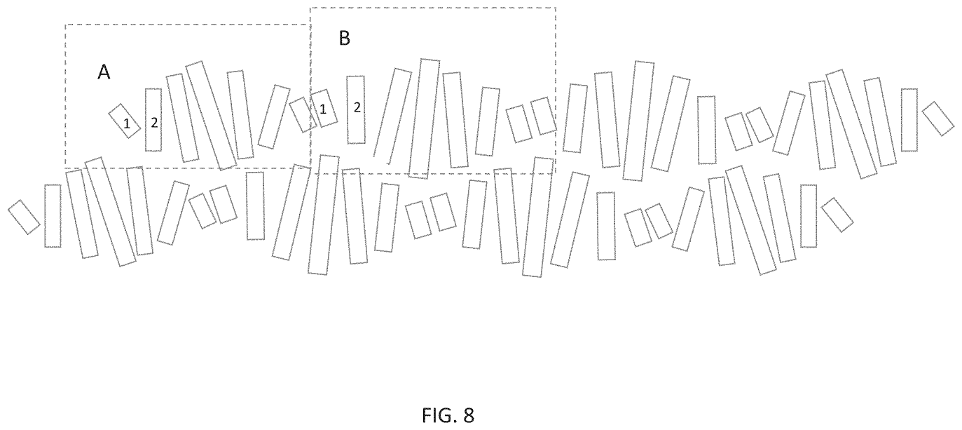

FIG. 7 depicts a texture obtained by reproducing the repeat pattern of the FIG. 6 in a series of rows. The rotation of the individual elements in a pattern with regard to one another reduces the amount of gloss generated. When the texture of the FIG. 7 is viewed from one direction, it may still be seen that the pathways are separated from one another by a sinusoidal pathway. There may be no smooth linear pathway when viewed from another direction as noted in the FIG. 1. If a linear pathway does exist it will be a jagged linear pathway, i.e., the inclination of some of the features will protrude into the field of view thus preventing the formation of a completely unobstructed pathway.

In another embodiment, it is desirable for the features in each pattern to be inclined at different angles (with respect to one another) relative to the inclination of the same features in the neighboring pattern. For example, in FIG. 8 the elements labelled 1 and 2 in pattern A are separated by a different angle that the elements labelled 1 and 2 in pattern B. All elements in the pattern A are separated from their neighboring elements by different angles than the corresponding elements in the pattern B. In short, each pattern will have comprise features that are inclined at different angles relative to one another when compared with the angles that separate the corresponding features in a neighboring pattern. If an average angle of inclination were to be calculated for each pattern (by adding the angle of inclination between immediate neighboring features in a pattern and dividing it by the number of features in the pattern), then the average angle of inclination for each pattern would be different from every other pattern. The greater the variation in the angle of inclination between neighboring patterns, the greater the reduction in constructive interference and consequently the gloss and glare.

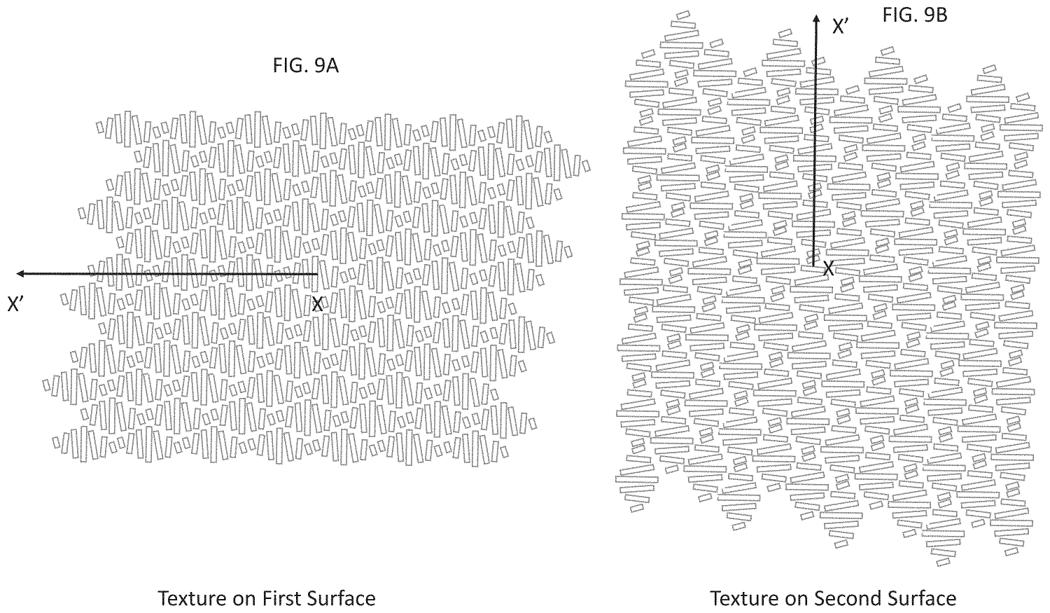

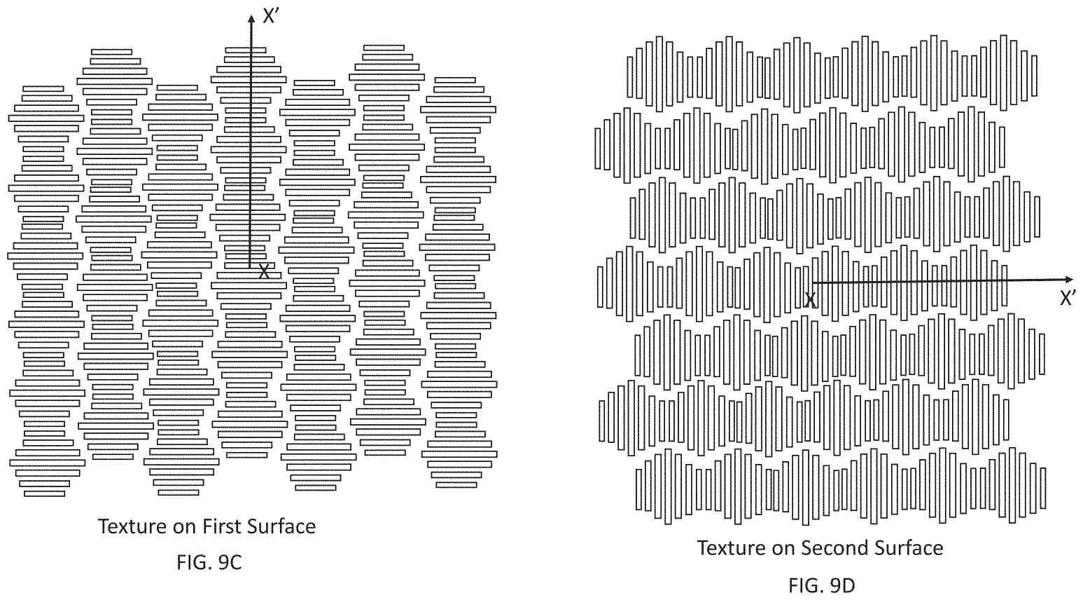

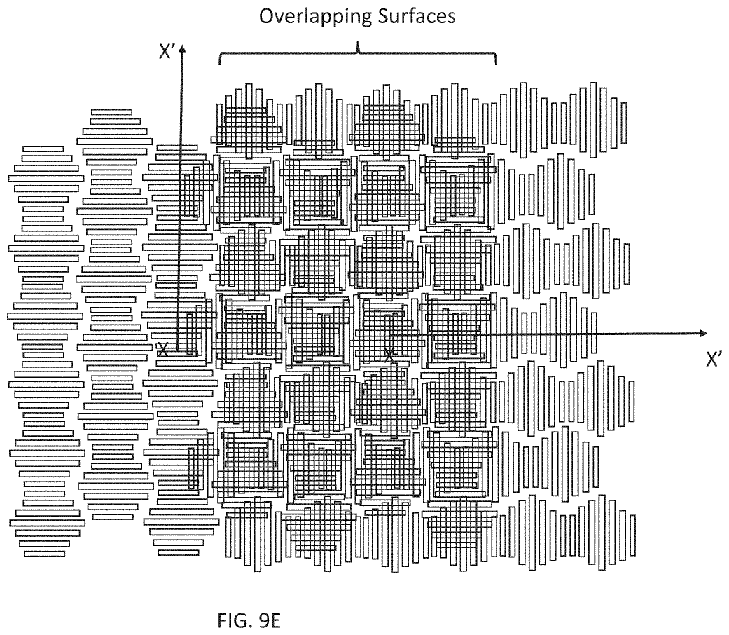

In another embodiment, a film containing the pattern on a first surface may have an identical pattern on an opposing second surface except that the pattern on the opposing second surface is rotated with respect to the pattern on the first surface. FIG. 9A shows the texture on the first surface while FIG. 9B shows the texture on the second surface. In the FIG. 9A, the axis of the pattern represented by line XX′ is horizontal, while in the FIG. 9B, the axis of the pattern represented by the line XX′ is vertical. The rotation of one pattern with respect to the other will also reduce constructive interference producing less gloss and glare. While the angle between the axis XX′ in the FIGS. 9A and 9B are 90 degrees apart, any angular difference between the two axis will reduce the glare and gloss. The reduction in glare and gloss is dependent upon the average angle of inclination of each pattern. When the average angle of inclination of the elements of a pattern is zero, then the glare will reach a minimum value at 90 degrees for a film having patterns on opposing surfaces.

This manner of reducing the gloss and glare can also be achieved by using the basic pattern shown in the FIG. 5. This is depicted in the FIGS. 9C and 9D. In the FIG. 9C, the axis of the pattern represented by line XX′ is vertical, while in the FIG. 9D, the axis of the pattern represented by the line XX′ is horizontal. The reduction in glare and gloss is dependent upon the average angle of inclination of the elements of each pattern. When the average angle of inclination of the elements of a pattern is zero (i.e., all elements are parallel to each other as in the FIGS. 9C and 9D), then the glare will reach a minimum value at 90 degrees for a film having patterns on opposing surfaces. The feature density may also be changed if desired. Pattern density may also be changed.

One such film may be seen in the FIG. 9E, where two patterns having their axis inclined at 90 degrees are superimposed on each other.

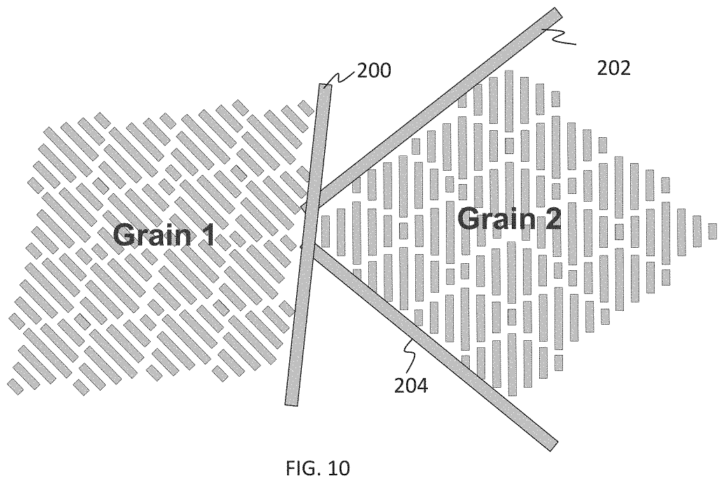

The FIG. 10 depicts another embodiment that may be used to control gloss and glare. In this embodiment, neighboring groups of patterns are rotated with respect to each other. The patterns of features may be arranged in groups of 4 to 20 (also referred to herein as grains) and then rotated with regard to each other. Spaces between the groups may be filled with partial patterns and with other convenient shapes that will facilitate bioadhesive control while at the same time reducing constructive interference.

In the FIG. 10, Grain 1 represents a collection of 20 patterns that is oriented in a first direction. Grain 2 represents a collection of 20 patterns and is rotated with respect to Grain 1. The rotation of Grain 2 with respect to Grain 1 promotes a reduction in glare and gloss; i.e., it is oriented in a second direction. The patterns are as defined above i.e., they have individual features that are arranged to have a sinusoidal path when viewed in a first direction and a linear path when viewed in a second direction.

The rotation of these grains with respect to each other however causes open spaces (as a result of geometrical mismatching) that are unfilled with the pattern. The absence of the pattern or a feature on the surface may change the bioadhesive control capabilities that the film is originally designed for. The may not be desirable. One way to overcome the lack of bioadhesive control, while at the same time reducing glare and gloss entails filling in regions of grain mismatch with partial patterns or with other geometrical features that facilitate a retention of bioadhesive control.

Another way to accomplish maintaining both bioadhesive control and gloss control is to use patterns whose features have different dimensions than those used in the grains. By using these differently sized features in groups of 4 to 20, the regions of grain mismatch can be filled in.

In the FIG. 10, the region between Grain 1 and Grain 2 is filled in with rectangular elongated features 200, 202 and 204 that are larger in size than any of the individual features seen in the grains themselves. Because these features are larger in size than the regular features used in the patterns of Grain 1 and Grain 2, these features can provide the film or article with reinforcement in a manner similar to the reinforcement provided by fibers to a composite. Since the orientation of these elongated features is different from those of the features of the patterns, they also facilitate reducing the glare and gloss.

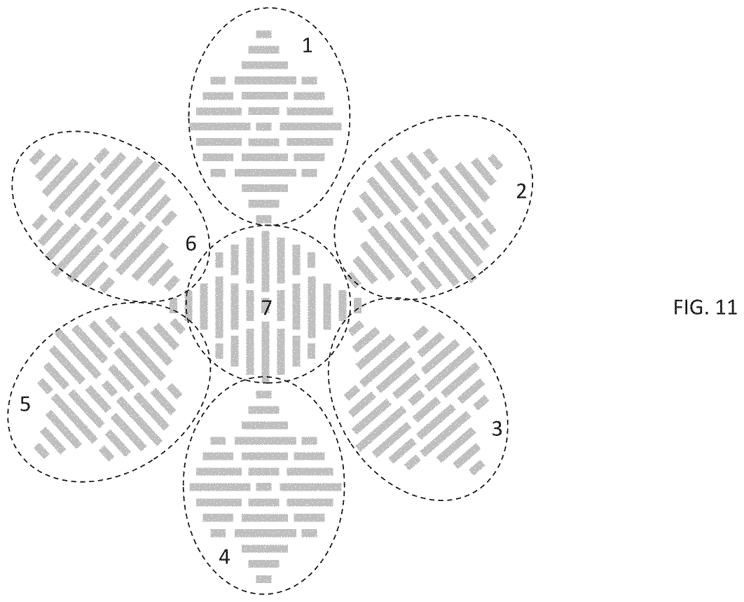

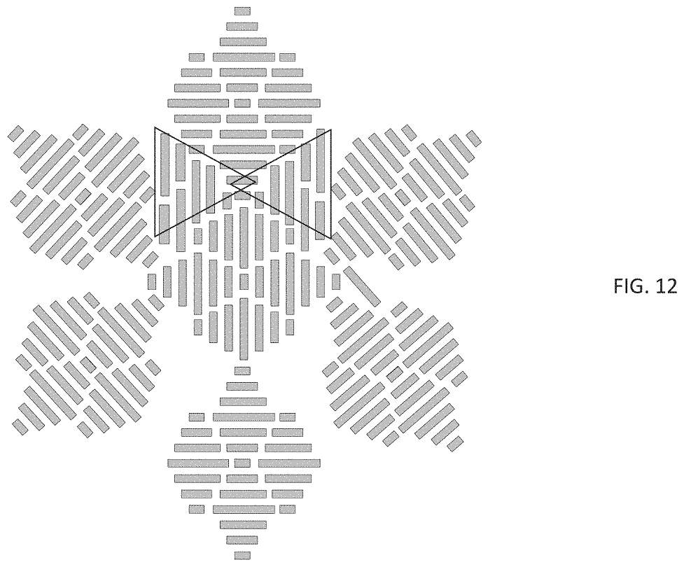

FIGS. 11 and 12 depict one exemplary embodiment of how regions between differently oriented grains may be filled in with partial patterns to accommodate bioadhesive control as well as gloss control. The FIG. 11 depicts an arrangement of groups of patterns to control glare. There are seven groups of patterns (seven grains) each comprising 4 patterns (of the basic pattern depicted earlier in the FIG. 5). Grains 1-6 are arranged on the periphery of Grain 7, which is located at the center of the configuration. Each of the grains is surrounded by a dotted ellipse or circle and each ellipse or circle is numbered for identification purposes. Each of the Grains 1-6 have a different orientation from Grain 7. Grains on the opposite sides of Grain 7 may have orientations that are very close to each other or alternatively, identical with each other.

This arrangement leaves a variety of open spaces between the grains that need to be filled in. Spaces between grains may be filled in with space filling features or space filling patterns.

The space filling features have different cross-sectional shapes from the cross-sectional shapes of the patterns that are used to texture the surface. Space filling patterns are patterns that have at least one shape having a cross-sectional geometry that is different from the cross-sectional geometry of the features that are used in the patterns. Space filling patterns may also be partial patterns. The space filling patterns will therefore be a) at least of a different size than the regular patterns that are used to texture the surface; b) at least of a different shape

The FIG. 12 shows one manner of filling such open spaces with partial patterns. In the FIG. 12, the open spaces between Grains 1 and 6 and between Grains 1 and 2 are filled in with partial patterns as depicted within the dotted triangles. The partial patterns comprise the rectangular features as shown previously in the FIG. 5, which is the regular base pattern deployed across the surface.

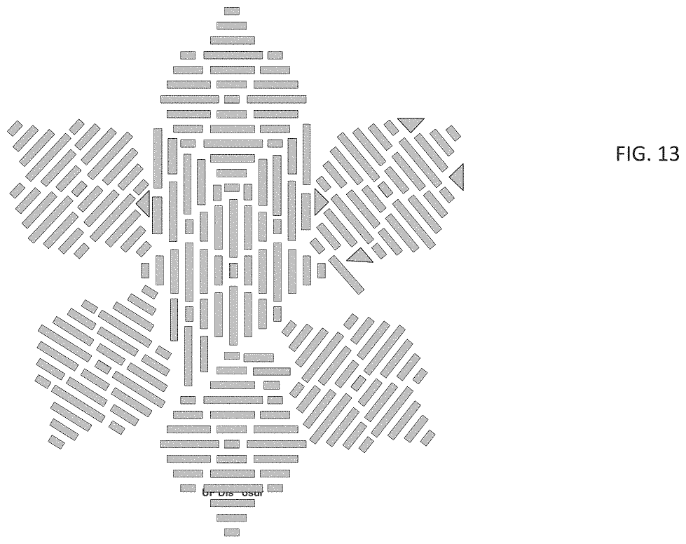

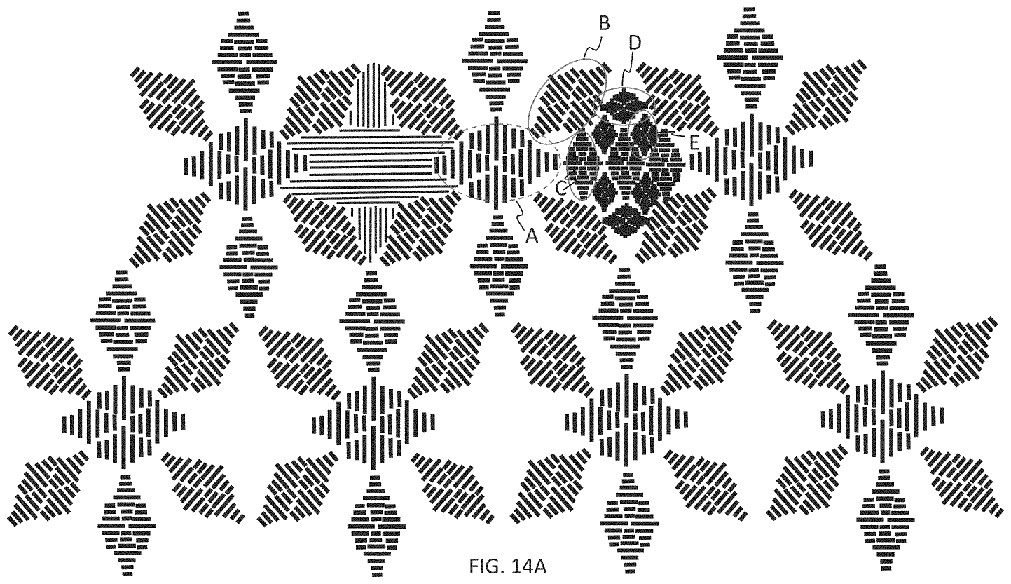

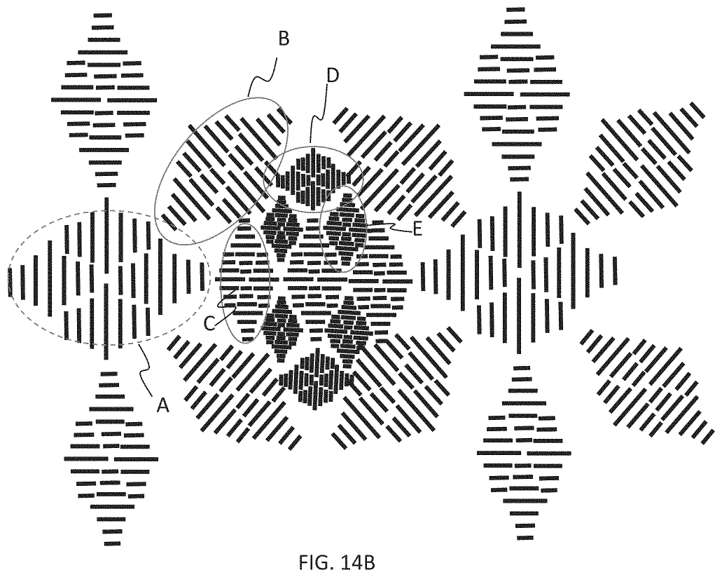

FIG. 13 depicts another texture where the spaces between Grains 1 and 6 and between Grains 1 and 2 are filled with partial patterns and triangles. Space filling features with other cross-sectional geometries may also be used such as squares, rhombus, parallelograms, circles, ellipses, polygons, or combinations thereof. The space filling features may have regular or irregular shapes. Combinations of irregular shapes may be used.

The space filling features are generally used in amounts of less than 5 percent of the total surface area covered by the patterns. Patterns that contain space filling shapes are termed space filling patterns. The space filling pattern contains at least one feature that has a different cross-sectional area from the remainder of features used in the patterns. For example, a pattern that has a triangle in addition to a plurality of rectangular features (that form the main pattern) may be considered to be a space filling pattern. Space filling patterns may comprise triangles, squares, circles, ellipsoids, parallelograms, rhomboids, hexagons, pentagons and other polygonal shapes. Irregular shaped features may also be used in such space filling patterns.

FIG. 14A depicts another texture where patterns having different sizes may be used. As noted above, in order to reduce glare and gloss, neighboring patterns may have a different orientation. In additional to different orientations, patterns having different sizes may also be used. Groups of patterns from 4 to 20 may be used in the patterns of different sizes. Thus in addition to patterns of different orientations, patterns having different sizes and features with different geometries may also be used to reduce glare. The patterns used to fill in spaces between patterns of different orientations may be larger or smaller than the main patterns.

In the FIGS. 14A and 14B (FIG. 14B is just an enlarged version of a portion of FIG. 14A), Grain A of a first size may be disposed on a surface where bioadhesion control and/or flow control is desired along with reduced gloss and glare. Grain A contains 4 patterns and has a first orientation. Grain B is then disposed next to Grain A at a different orientation from Grain A. Grain B also contains 4 patterns that have a second size (smaller than the first size of patterns in Grain A) that have a second orientation different from the first orientation of patterns in Grain A. Grain C is then disposed next to Grain B at a different orientation (a third orientation) from Grain B. The patterns in Grain C (also 4 in number) are smaller than the patterns in Grain B. Grain D is then disposed next to Grain C at a different orientation (a fourth orientation) from Grain C. The patterns in Grain D (also 4 in number) are smaller than the patterns in Grain C. Grain E is then disposed next to Grain D at a different orientation (a fifth orientation) from Grain D. The patterns in Grain E (also 4 in number) are smaller than the patterns in Grain D.

In an embodiment, when patterns of different sizes (but having the same overall shape) are used next to each other in order to disperse incident light, it is desirable for each succeeding smaller pattern to have a size that is decreased from the largest pattern size as dictated by a serial progression. Series progressions may be used to vary pattern size and orientation in order to reduce constructive interference and hence glare and gloss. Examples of series progressions may include geometrical progressions, arithmetical progressions, exponential progressions, or the like.

A geometric progression, also known as a geometric sequence, is a sequence of numbers where each term after the first is found by multiplying the previous one by a fixed, non-zero number called the common ratio. For example, the sequence 2, 6, 18, 54, . . . is a geometric progression with common ratio 3. Similarly 10, 5, 2.5, 1.25, is a geometric sequence with common ratio 1/2.

Examples of a geometric sequence are powers r^{k} of a fixed number r, such as 2k and 3k.

The general form of a geometric sequence is:

a, ar, ar², ar³, ar⁴, . . . where r≠0 is the common ratio and a is a scale factor, equal to the sequence's start value.

For example, in a pattern having a scale factor of a=1 and a fixed ratio of ½, if the largest feature in a pattern A has a unit length of 1 unit, then the pattern B next to it that is used to fill up unoccupied space will have a unit length of ½ unit. Pattern B will have a different orientation from pattern A. The rest of the features in the pattern B will have sizes based on the dimension of ½ unit. In other words, the remaining features of the pattern B, will have a length less than ½ unit.

If there is still unoccupied space that is to be filled up, it can be filled up with another pattern B or alternatively, if the space is smaller than the pattern B, it can be filled up with a pattern C, oriented differently from pattern B, and having its largest feature have a length of ¼ unit. The remaining features in the pattern C will be smaller than ¼ unit. In this manner, the next successive pattern D, will have its largest feature be ⅛ unit in length and pattern E will have its largest feature be 1/16 unit in length.

The decrease in size from the largest pattern to the smallest pattern does not progress along any particular direction, but progresses in a manner where open spaces are filled with the next smaller pattern size.

While the set of patterns in the FIG. 14B are decreased in size using a geometrical series approximation for the largest feature, other series approximations may be used to vary pattern size and orientation in order to reduce constructive interference and hence glare and gloss. Other functions may include binomial series, arithmetical series, exponential series, or the like.

An arithmetic progression (AP) or arithmetic sequence is a sequence of numbers such that the difference between the consecutive terms is constant. For instance, the sequence 5, 7, 9, 11, 13, 15, . . . is an arithmetic progression with common difference of 2.

In an embodiment, it may be desirable for each succeeding pattern to have a size that is increased from the smallest pattern size as dictated by an arithmetical serial progression.

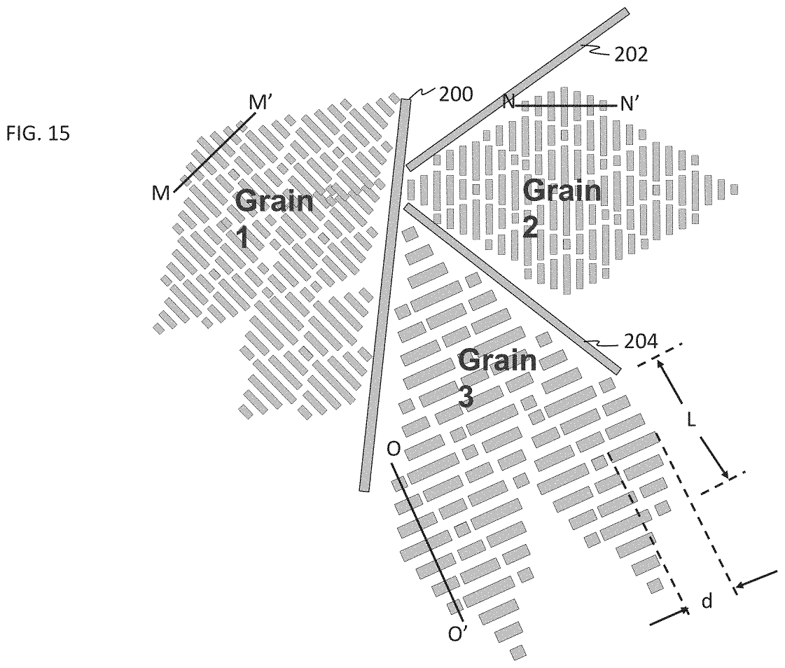

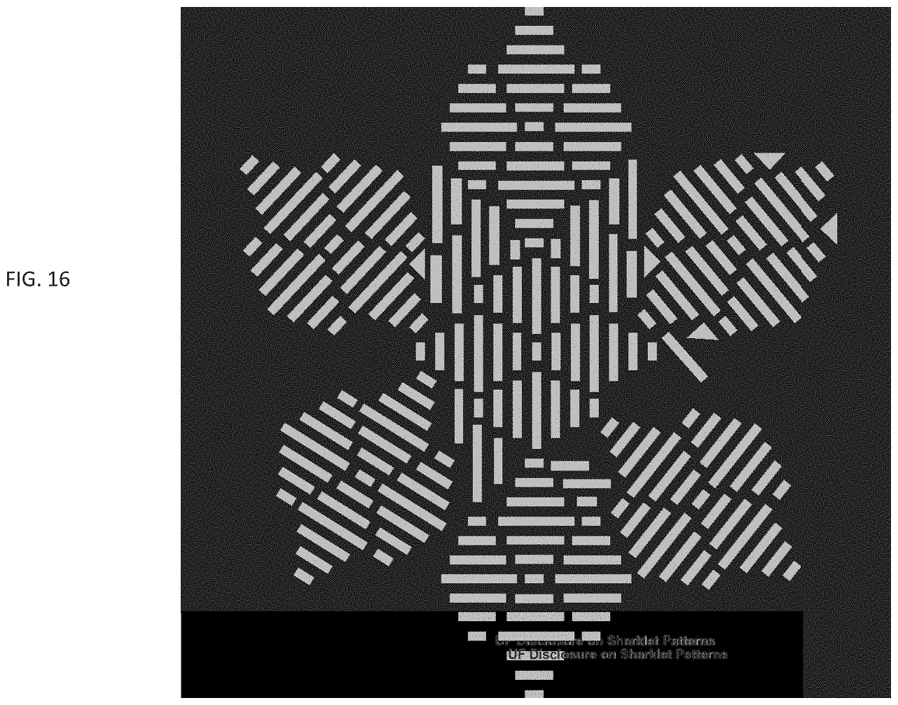

The FIG. 15 depicts another exemplary embodiment where the density of features is varied in order to accommodate a pattern whose orientation is different from that of the other patterns on the textured surface. The FIG. 15 depicts three grains—Grain 1, Grain 2 and Grain 3 each of which have a different orientation. The orientation may be determined by an axis that bisects one pattern in every given grain. For example, the axis MM′ determines the orientation of Grain 1, while axis NN′ determines the orientation of Grain 2 and 00′ determines the orientation of Grain 3. As may be seen from the FIG. 15, each grain is oriented differently from the other grain on the textured surface. It may also be noted that Grain 1 and Grain 2 are constructed of features that have the same size. In other words, the feature density in Grain 1 and Grain 2 are the same. However, because of their different orientation, any other grains that are to be disposed on the same surface in order to fill vacant spaces would have to be of a different size. In other words, the feature density for Grain 3 will have to be different from that of Grain 1 or Grain 2.

In order to fill the space between Grain 1 and Grain 2, the feature density of Grain 3 is decreased. The pattern of Grain 3 is therefore elongated relative to the pattern of Grain 1 and Grain 2. The elongation of the pattern of Grain 3 results in patterns having a greater aspect ratio than those of Grain 1 and Grain 2. The aspect ratio of a pattern (which comprises a grouping of features that are different from one another) is the length “L” of a pattern divided by the width “d” of the pattern. The aspect ratio of the pattern can vary from 2:1 to 20:1.

FIG. 15 therefore shows several grains of different orientations where one or more patterns that form the grain may have a different aspect ratio from the patterns in neighboring grains. The grains have groups of 4 to 20 patterns. Neighboring grains have different orientations as determined by their axes, where the axis is a bisector of a pattern in a grain. 3 or more grains may abut each other with each grain having a different orientation and where at least one of the grains has patterns that has a different aspect ratio from the aspect ratio of one or more of the neighboring patterns. In an embodiment, the feature density of the patterns in one grain are different from the feature density of the patterns in a neighboring grain. In an embodiment, the feature density of the patterns in one grain are different from the feature density of the patterns in every neighboring grain.

Other gloss reducing methods may include roughening the surface of the features, adding light absorbing fillers to the features, and the like.

Surface roughening may be accomplished by sand blasting the surface, etching the surface, plasma treating the surface, and the like. Etching can include mechanical etching, chemical etching, and the like.

In an embodiment, the surface may be coated with features that can scatter light in numerous directions thus preventing the occurrence of constructive interference. Domains of particles can be disposed on the features using chemical vapor deposition, plasma vapor deposition, atomic vapor deposition, and the like.

Metals islands from metals such as copper, aluminum, tin, platinum, gold, and the like, may be disposed features to reduce glare and gloss caused by the conductive interference. Islands of metal oxides may also be deposited by vapor or liquid deposition methods. Suitable metal oxides that may be used to form the islands include silicon dioxide, aluminum oxide, zirconium oxide, titanium dioxide, or a combination thereof.

The features displayed in the FIGS. 5 to 15 may be manufactured from organic polymers, metals, ceramics, or combinations thereof.

Organic polymers used in the spaced features and/or the surface can be may be selected from a wide variety of thermoplastic polymers, blend of thermoplastic polymers, thermosetting polymers, or blends of thermoplastic polymers with thermosetting polymers. The organic polymer may also be a blend of polymers, copolymers, terpolymers, or combinations comprising at least one of the foregoing organic polymers. The organic polymer can also be an oligomer, a homopolymer, a copolymer, a block copolymer, an alternating block copolymer, a random polymer, a random copolymer, a random block copolymer, a graft copolymer, a star block copolymer, a dendrimer, a polyelectrolyte (polymers that have some repeat groups that contain electrolytes), a polyampholyte (a polyelectrolyte having both cationic and anionic repeat groups), an ionomer, or the like, or a combination comprising at last one of the foregoing organic polymers. The organic polymers have number average molecular weights greater than 10,000 grams per mole, preferably greater than 20,000 g/mole and more preferably greater than 50,000 g/mole.

Examples of thermoplastic polymers that can be used in the polymeric material include polyacetals, poly acrylics, polycarbonates, polyalkyds, polystyrenes, polyolefins, polyesters, polyamides, polyaramides, polyamideimides, polyarylates, polyurethanes, epoxies, phenolics, silicones, polyarylsulfones, polyethersulfones, polyphenylene sulfides, polysulfones, polyimides, polyetherimides, polytetrafluoroethylenes, polyetherketones, polyether ether ketones, polyether ketone ketones, polybenzoxazoles, polyoxadiazoles, polybenzothiazinophenothiazines, polybenzothiazoles, polypyrazinoquinoxalines, polypyromellitimides, polyguinoxalines, polybenzimidazoles, polyoxindoles, polyoxoisoindolines, polydioxoisoindolines, polytriazines, polypyridazines, polypiperazines, polypyridines, polypiperidines, polytriazoles, polypyrazoles, polycarboranes, poly oxabicyclononanes, polydibenzofurans, polyphthalides, polyacetals, polyanhydrides, polyvinyl ethers, polyvinyl thioethers, polyvinyl alcohols, polyvinyl ketones, polyvinyl halides, polyvinyl nitriles, polyvinyl esters, polysulfonates, polysulfides, polythioesters, polysulfones, polysulfonamides, polyureas, polyphosphazenes, polysilazanes, polypropylenes, polyethylenes, polyethylene terephthalates, polyvinylidene fluorides, polysiloxanes, or the like, or a combination thereof.

Examples of polyelectrolytes are polystyrene sulfonic acid, polyacrylic acid, pectin, carrageenan, alginates, carboxymethylcellulose, polyvinylpyrrolidone, or the like, or a combination thereof.

Examples of thermosetting polymers suitable for use as hosts in emissive layer include epoxy polymers, unsaturated polyester polymers, polyimide polymers, bismaleimide polymers, bismaleimide triazine polymers, cyanate ester polymers, vinyl polymers, benzoxazine polymers, benzocyclobutene polymers, acrylics, alkyds, phenol-formaldehyde polymers, novolacs, resoles, melamine-formaldehyde polymers, urea-formaldehyde polymers, hydroxymethylfurans, isocyanates, diallyl phthalate, triallyl cyanurate, triallyl isocyanurate, unsaturated polyesterimides, or the like, or a combination thereof.

Examples of blends of thermoplastic polymers include acrylonitrile-butadiene-styrene/nylon, polycarbonate/acrylonitrile-butadiene-styrene, acrylonitrile butadiene styrene/polyvinyl chloride, polyphenylene ether/polystyrene, polyphenylene ether/nylon, polysulfone/acrylonitrile-butadiene-styrene, polycarbonate/thermoplastic urethane, polycarbonate/polyethylene terephthalate, polycarbonate/polybutylene terephthalate, thermoplastic elastomer alloys, nylon/elastomers, polyester/elastomers, polyethylene terephthalate/polybutylene terephthalate, acetal/elastomer, styrene-maleic anhydride/acrylonitrile-butadiene-styrene, polyether etherketone/polyethersulfone, polyether etherketone/polyetherimide polyethylene/nylon, polyethylene/polyacetal, or the like.

Polymers that can be used also include biodegradable materials. Suitable examples of biodegradable polymers are as polylactic-glycolic acid (PLGA), poly-caprolactone (PCL), copolymers of polylactic-glycolic acid and poly-caprolactone (PCL-PLGA copolymer), polyhydroxy-butyrate-valerate (PHBV), polyorthoester (POE), polyethylene oxide-butylene terephthalate (PEO-PBTP), poly-D,L-lactic acid-p-dioxanone-polyethylene glycol block copolymer (PLA-DX-PEG), or the like, or a combination thereof.

Suitable metals for manufacturing the features include transition metal substrates, alkaline earth metal substrates, alkali substrates, or a combination thereof. Suitable metals include iron, copper, titanium, aluminum, vanadium, gold, silver, zinc, molybdenum, nickel, cobalt, silicon, gallium, indium, thallium, or the like, or a combination thereof.

Suitable ceramics for manufacturing the features include metal oxides, metal carbides, metal nitrides, metal borides, metal silicides, metal oxycarbides, metal oxynitrides, metal boronitrides, metal carbonitrides, metal borocarbides, or the like, or a combination thereof. Examples of ceramics that may be used as the substrate include silicon dioxide, aluminum oxide, titanium dioxide, zirconium dioxide, indium tin oxide, antimony tin oxide, cerium oxide, cadmium-oxide, titanium nitride, silicon nitride, aluminum nitride, titanium carbide, silicon carbide, titanium niobium carbide, stoichiometric silicon boride compounds (SiBₙ, where n=14, 15, 40, and so on) (e.g., silicon triboride, SiB₃, silicon tetraboride, SiB₄, silicon hexaboride, SiB₆, or the like), or the like, or a combination thereof.

The pattern designs disclosed herein minimize gloss and glare by reducing constructive interference. The pattern can also be disposed on the surface of a variety of miscellaneous items such as, for example, clothing and accessories, sunglass lenses, frames of sunglasses, eye glass lenses, surfaces and frames of aquariums, outdoor clothing, water resistant jackets, coats, sports clothing, swimsuits, wetsuits, surfboards, outdoor equipment, tents, lanterns, lamps, tickets (e.g., to sporting events, airline tickets, train and ship tickets), shirt and dress collars, textile surfaces that contact armpits and other private parts of the body, and the like. Such surfaces can be marketed as being antimicrobial surfaces.

The pattern can also be disposed on the surfaces of camping equipment (e.g., tents, poles, lamps, and the like), camping gear, sports equipment (e.g., parachutes, parachute rigs, parachute bags, insides and outsides of shoes, insoles, and the like), and the like. Such equipment can be marketed as water resistant equipment that deters microorganism aggregation. It can also be marketed as deterring the buildup of odor in shoes and underwear.

The pattern can also be disposed on the surfaces of marine vessels and other devices that contact water. For example, it can be used on boat hulls, intake and outlet pipes for industrial and power plants, drilling rig for underwater surfaces, fish tanks and aquariums, boat surfaces (above the hull), bilge tanks, water treatment plants and pumping station surfaces—any surface inside such a water treatment plant and pumping station where organism growth and colonization is an issue. The pattern can be disposed on the surfaces of bags used to grow algae, for example, it can be used on the surface of a bag used to grow any microorganism but prevent attachment of the microorganism onto the surface of bag (medical or marine—e.g., blood bags where it is desirable to deter organism attachment to bag). Alternatively, by varying the surface texture or the size of the texture dimensions, it can be used on the surface of a bag used to grow any microorganism and encourage attachment of the microorganism to surface of the bag (e.g., a stem cell culture where it is desirable to encourage growth and attachment to surface).

The pattern can also be disposed on a variety of other items: bags, handbags, garbage bags, bags that are used for carrying tissue, fluids from living beings, waste and other byproducts from living beings, and the like. Examples of tissue, fluids, waste from living beings are urine, blood, saline, glucose, feces, fluids from the mucous membranes, and the like.

The pattern can also be used on the surfaces of body parts that are used in surgeries such as, for example, in a colostomy, and the like. It can also be used in replacement joints, plates, tendon and ligament ends for enhanced tissue adaptation, vascular implants, grafts, shunts, access, and the like. The pattern may also be used on the inner and outer surfaces of periodontal dressings; intravenous catheters and ports; foley catheters; surfaces in contact with tissues such as, for example, plates; adhesive tapes, patches, bandages, and the like; electronic leads; dental implants; orthodontia devices; iols (intraocular lenses); hydrogel films for tissue enhancement, skin grafting, isolation of bacteria from tissues; heart-lung machine surfaces to reduce infection, clotting/thrombosis, enhance flow; tissue constructs for organ/tissue genesis; dialysis machine components, tubing and control panels; cochlear/otolaryngology implants and electronic devices; pace maker leads and body; fibrillator leads and body; heart valve flow surfaces and fixation surfaces; spinal implants; cranial/facial implants; biomedical instruments such as, for example, heart valves; scalpels; tongs; forceps; saws; reamers; grippers; spreaders; pliers; hammers; drills; laryngoscopes; bronchoscopes; oesophagoscopes; stethoscopes, mirrors, oral/ear speculum, xray plates/frames, xray device surfaces, magnetic resonance imaging (MRI) surfaces, echo cardiogram surfaces, cat-scan surfaces, scales, clipboards, and the like.

The pattern can be disposed on hospital surfaces. For example, it can be used as a film to be applied to surfaces that can be readily replaced between surgeries. For example, it can be applied to such surfaces as listed below using electrostatic adhesion, mechanical interlocking or adhesives. The film can be used on table tops, MRI/CAT scan surfaces, X-ray surfaces, scales, operating tables, door push panels, devices or articles that are contacted by human beings such as, for example, light switches, control panels, beds, incubators, monitors, remote controls, call buttons, door push bars, preparation surfaces, instrument trays, pharmacy surfaces, pathology tables, outside surfaces of bed pans, identification surfaces on walls, clothing/protective personal wear, gloves, cling films to attach temporary in public rest rooms/areas, baby changing cling films, films for attaching to bottoms of purses/bags/suitcases, biomedical packaging, such as the outside surface of sterilized packaging; vacuum formed trays/films, cling films for short and long term use, clean room surfaces, such as, for example, those used for the semiconductor or biomedical industry, table tops, push bars, door panels, control panels, instruments, entrance/exit points, food industry, including for packaging, food preparation surfaces, counter tops, cutting boards, trays, entrance/exit points, switches, control panels, scales, packaging equipment operator contact points, marine industry, exterior surfaces of marine vessels including ships, bilge tanks, gray water tanks, water inlet/outlet pipes, power drive systems, propellers, jet ports, water treatment plants including pumping stations, inlet/outlet pipes, control panel surfaces, laboratory surfaces, power plants, inlet/outlet pipes, control surfaces, airline industry, trays on seatbacks, entry/exit push surfaces, bathroom doors, service carts, arm rests, furniture industry, children's cribs, handles on exercise equipment, exercise equipment contact surfaces, changing tables, high chairs, table tops, food prep surfaces, transportation industry, ambulances, buses, public transit, swimming pools.

In an embodiment, a method of disposing a pattern on the surface of an article comprises disposing a first plurality of spaced features; the spaced features arranged in a plurality of groupings; the groupings of features comprising repeat units; the spaced features within a grouping being spaced apart at an average distance of about 1 nanometer to about 500 micrometers; each feature having a surface that is substantially parallel to a surface on a neighboring feature; each feature being separated from its neighboring feature; and wherein the groupings of features being arranged with respect to one another so as to define a tortuous pathway; and further wherein a) the longitudinal axis of neighboring features are not parallel to one another and are inclined at varying angles of 5 to 85 degrees with respect to one another; b) the patterns are grouped together in groups of 4 to 20 with each neighboring group having a different axis of orientation; c) opposing surfaces of the article each have the texture, where the texture on one surface is oriented differently from the texture on the opposing surface; d) the features of the texture have roughened surfaces; and/or e) the features contain fillers that can absorb visible light.

The method for disposing the pattern includes injection molding, blow molding, vacuum forming, and the like.

It is to be understood that while the invention has been described in conjunction with the preferred specific embodiments thereof, that the foregoing description as well as the examples, which follow are intended to illustrate and not limit the scope of the invention. Other aspects, advantages and modifications within the scope of the invention will be apparent to those skilled in the art to which the invention pertains.

1. An article comprising:
a pattern comprising a first plurality of spaced features; the spaced features arranged in a plurality of groupings; the groupings of features comprising repeat units; the spaced features within a grouping being spaced apart at an average distance of about 1 nanometer to about 500 micrometers; each feature having a surface that is substantially parallel to a surface on a neighboring feature; each feature being separated from its neighboring feature; and wherein the groupings of features being arranged with respect to one another so as to define a tortuous pathway; and further wherein
a) the longitudinal axis of neighboring features are not parallel to one another and are inclined at varying angles of 5 to 85 degrees with respect to one another;
b) the patterns are grouped together in groups of 4 to 20 with each neighboring group having a different axis of orientation;
c) opposing surfaces of the article each have the texture, where the texture on one surface is oriented differently from the texture on the opposing surface;
d) the features of the texture have roughened surfaces; and/or
e) the features contain fillers that can absorb visible light.

2. The article of claim 1, wherein the plurality of spaced feature extend outwardly from a surface.

3. The article of claim 2, wherein the plurality of spaced features has a similar chemical composition to the surface.

4. The article of claim 2, wherein the plurality of spaced features has a different chemical composition from that of the surface.

5. The article of claim 2, wherein the plurality of spaced features is applied to the surface in the form of a coating.

6. The article of claim 1, wherein the plurality of spaced features are projected into a surface of the article.

7. The article of claim 1, wherein the groupings of features are arranged with respect to one another so as to define a linear pathway or a plurality of channels.

8. The article of claim 1, wherein the tortuous pathway is defined by a sinusoidal curve.

9. The article of claim 1, wherein the tortuous pathway is defined by a spline function.

10. The article of claim 1, where patterns comprising (a) are disposed on opposing surfaces of the article.

11. The article of claim 10, where the patterns on one surface are inclined with respect to patterns on an opposing surface.

12. The article of claim 1, where patterns comprising (b) are separated by space filling patterns.

13. The article of claim 1, where the space filling pattern may comprise regular or irregular features.

14. The article of claim 1, where the space filling pattern may contain at least one feature that is not contained in other patterns and where the at least one feature has a different cross-sectional shape from the cross-sectional shape of other features contained in the regular patterns.

15. The article of claim 1, wherein patterns comprising (b) each neighboring group has a different axis of orientation

16. The article of claim 15, where each neighboring group that has a different axis of orientation is successively decreased in size according to a geometric progression.

17. The article of claim 15, where each neighboring group that has a different axis of orientation is successively increased in size according to an arithmetic progression.

18. A method comprising:
disposing on a surface a pattern comprising a first plurality of spaced features; the spaced features arranged in a plurality of groupings; the groupings of features comprising repeat units; the spaced features within a grouping being spaced apart at an average distance of about 1 nanometer to about 500 micrometers; each feature having a surface that is substantially parallel to a surface on a neighboring feature; each feature being separated from its neighboring feature; and wherein the groupings of features being arranged with respect to one another so as to define a tortuous pathway; and further wherein
a) the longitudinal axis of neighboring features are not parallel to one another and are inclined at varying angles of 5 to 85 degrees with respect to one another;
b) the patterns are grouped together in groups of 4 to 20 with each neighboring group having a different axis of orientation;
c) opposing surfaces of the article each have the texture, where the texture on one surface is oriented differently from the texture on the opposing surface;
d) the features of the texture have roughened surfaces; and/or
e) the features contain fillers that can absorb visible light.
